(19) United States
(12) Patent Application Publication
(10) Pub. No.: US 2024/0000719 A1
(43) Pub. Date: Jan. 4, 2024

(54) FORMULATIONS

(71) Applicant: UCB BIOPHARMA SRL, BRUSSELS (BE)

(72) Inventors: SARAH MARQUETTE, BRUSSELS (BE); JONATHAN ELEUTHÈRE MAURICE GOOLE, BRUSSELS (BE); EMERIC CARLIER, BRUSSELS (BE)

(21) Appl. No.: 18/039,498

(22) PCT Filed: Nov. 29, 2021
(86) PCT No.: PCT/EP2021/083401

§ 371 Date: May 31, 2023

(30) Foreign Application Priority Data
Dec. 1, 2020 (GB) 2018889.2

(51) Int. Cl. A61K 9/70; A61K 47/26; A61K 9/14; A61K 47/10; A61K 47/34; A61K 47/18; A61K 9/19; A61K 9/00

(52) U.S. Cl. CPC A61K 9/70; A61K 47/26; A61K 9/146; A61K 47/10; A61K 47/34; A61K 47/183; A61K 9/19; A61K 9/0024; B33Y 70/00

(57) ABSTRACT

The invention relates to the field of pharmaceutical compositions comprising proteins as therapeutic agents. More particularly, it is directed to hot melt extrusion-produced antibody-containing filaments, implantable drug delivery devices made from these filaments and to methods of producing such filaments and devices. The hot melt extrusion-produced antibody-containing filaments and the devices obtained from the filaments according to the invention allow the delivery of the antibody over a certain period of time.

### FIELD OF INVENTION

The invention relates to the field of pharmaceutical compositions comprising proteins as therapeutic agents. More particularly, it is directed to hot melt extrusion-produced antibody-containing filaments, implantable drug delivery devices made from these filaments and to methods of producing such filaments and devices. The hot melt extrusion-produced antibody-containing filaments and the devices obtained from the filaments according to the invention allow the delivery of the antibody over a certain period of time.

### BACKGROUND OF THE INVENTION

The hot melt extrusion (HME) is widely described and implemented in the pharmaceutical field to produce drug-loaded printable filaments (Goyanes et al., 2015; Tiwari et al., 2016). HME is based on the melting of polymeric material that is extruded through a die to obtain a homogeneous drug-loaded filament. HME is a free-solvent process which may be easily scaled-up. However, this technique is based on the use of relatively high temperatures. Such temperatures may be usually reduced by adding a plasticizer, allowing the decrease of the glass transition temperature of the polymer. Another alternative to decrease the temperature of extrusion could be the use of thermoplastic polymers characterized by a low molecular weight (Fredenberg et al., 2011). HME was already investigated to develop protein-based formulations which were characterized by a controlled-release of the loaded active ingredient over time (Cossé et al., 2016; Duque et al., 2018; Ghalanbor et al., 2010).

HME can be used in combination with 3D printing (3DP) process, such as fused deposition modelling (FDM™). FDM process is currently an integrant part of the pharmaceutical field (Jamroz et al, 2018; Azad et al., 2020). This technology is an extrusion-based 3DP method which uses heat to melt a thermoplastic polymer filament to build an object in a layer-wise manner. The use of 3DP allows the production of any kind of shapes starting from a digital design (Norman et al., 2017). The main drawback remains the lack of pharmaceutical grade polymers that are available to be used in FDM, although Poly(lactic acid) (PLA) and polyvinyl alcohol (PVA) are commonly used as thermoplastic polymers to make drug-loaded printable filaments (Jamróz et al., 2018).

Poly(lactide-co-glycolide) (PLGA) is a well-known pharmaceutical grade polymeric material that is usually used to make injectable/implantable sustained-release DDS. PLGA could be extruded at low temperature, making it a good candidate for both HME and FDM processes. Protein-loaded PLGA implants have already been described using macromolecules such as ovalbumin (Duque et al., 2018), bovine serum albumin (Cossé et al., 2016) and lysozyme (Ghalanbor et al., 2010). The major challenge remains the stabilization of the protein during the extrusion.

It was shown that the solid state of the protein could be more advantageous to promote a higher stability as well as to make easier its addition into the polymeric matrix using HME process (Cossé et al., 2016; Mensink et al., 2017). However, the protein compounds usually used as models (i.e. OVA, BSA, lysozyme) to produce protein-loaded implants are characterized by low molecular weights in comparison with immunoglobulins for instance.

Therefore, there is still a need for further filaments and implantable drug delivery devices comprising large proteins, more particularly antibodies, with sustained-release properties, improving stability of antibodies (e.g. limiting antibody degradation during the production of the filament and then of the implantable drug delivery device), while keeping their activity (i.e. without impacting drastically their biological activity).

### SUMMARY OF THE INVENTION

In a first aspect, the present invention provides a filament for preparing an implantable drug delivery device, wherein the filament comprises or consists of at least one polymeric material, a plasticizer and an active ingredient, wherein said active ingredient is an antibody. The filament may further comprise at least one stabilizer, a buffering agent and/or a surfactant.

In a second aspect, the present invention relates to an implantable drug delivery device comprising or consisting of one or more layers made from a filament comprising or consisting of at least one polymeric material, a plasticizer and an active ingredient, wherein said active ingredient is an antibody. The filament may further comprise at least one stabilizer, a buffering agent and/or a surfactant.

In a third aspect, the present invention describes a 3D printed implantable drug delivery device obtained by 3D printing filaments comprising or consisting of at least one polymeric material, a plasticizer and an active ingredient, wherein said active ingredient is an antibody. The filament may further comprise at least one stabilizer, a buffering agent and/or a surfactant.

In a fourth aspect, the present invention provides a process for producing a filament for preparing an implantable drug delivery device, the process comprising the steps of:
a. preparing a liquid formulation comprising or consisting of the active ingredient, wherein said liquid formulation may further comprise at least one stabilizer, a buffering agent and/or a surfactant, wherein said active ingredient is an antibody,
b. freeze-drying or spray-drying the liquid formulation of step a. to obtain dry microparticles,
c. dispersing homogeneously the dry microparticles of step b. with a plasticizer and at least one polymeric material,
d. extruding the dispersion of step c. by hot melting extrusion (HME) to obtain a filament.

In a fifth aspect, the present invention relates to a process for producing an implantable drug delivery device, the process comprising the steps of:
a. loading the filament herein described into the print head of the 3D printer using a temperature above the glass transition temperature,
b. heating the build platform at a temperature below the glass transition temperature of the polymeric matrix;
c. depositing said heated filament through a nozzle to build the device from at least the first layer to the final top layer.

### Definitions

The term “dry microparticle” (dry microparticles in its plural form) refers to a dry “particle” of very small size (size typically of about 20 μm or below) (alternatively named “microparticles” or “microspheres”). Preferably the dry microparticle contains water below about 10%, usually below 5% or even below 3% by weight of the dry particles. A dry microparticle can typically be obtained by spray-drying and/or freeze-drying an aqueous solution or an aqueous emulsion. Alternatively, the term dry powder can be used.
The term “freeze-drying” also known as “lyophilization” refers to a process for obtaining a dry microparticle comprising at least three main steps: 1) lowering the temperature of the product to be freeze-dried to below freezing point (typically between -40 and -80° C.; freezing step), 2) applying a high-pressure vacuum (typically between 30 and 300 mTorr; first drying step) and 3) increasing the temperature (typically between 20 and 40° C.; second drying step).
The term “spray drying” refers to a process for obtaining dry microparticles comprising at least two main steps: 1) atomizing a liquid feed into fine droplets and 2) evaporating the solvent or water by means of a hot drying gas.
The term “slow-release” (herein alternatively named “sustained-release”) refers to the delivery of the active ingredient over days, weeks, months or even years. The typical slow-release profile for a protein-loaded polymeric microparticle is triphasic and consists of (i) an initial burst release (i.e. the release of an initial large amount of active ingredient), (ii) a lag phase (i.e. a phase during which very low amount or no product is released) and (iii) a release phase (i.e. a phase during which the release rate is stable) (Diwan et al., 2001 and White et al., 2013). An initial burst release of preferably no more than about 40% of the total amount of active ingredient will be deemed acceptable. Any initial burst release of no more than 30% will be called a “limited burst release”. The release of the antibody molecule should also be as complete as possible (i.e. total release as close as possible to 100% of the encapsulated antibodies), and preferably at least above 60%. One of the advantages of such a slow-release composition is that the composition will be administered less often to the patient.
The term “stability”, as used herein, refers to the physical, chemical, and conformational stability of the active ingredient (herein an antibody) in the filaments and drug delivery devices according to the present invention (and including maintenance of biological potency). Instability of the antibody may be caused by chemical degradation or aggregation of the antibody to form for instance higher order polymers, deglycosylation, modification of glycosylation, oxidation or any other structural modification that reduces the biological activity of the formulated antibody. The term “stable” refers to filaments or drug delivery devices in which the active ingredient (herein an antibody) essentially retains its physical, chemical and/or biological properties during manufacturing and upon storage. In order to measure the antibody stability in a formulation, various analytical methods are well within the knowledge of the skilled person (see also the example section). Various parameters can be measured to determine stability of the filaments or of the 3DP devices (in comparison with the initial data), such as (and not limited to): 1) no more than about 15% of alteration of the monomeric form of the antibody, or 2) no more than 15% High Molecular Weight Species (HMW or HMWS; also herein referred to as aggregates).
The term “buffer” or “buffering agent”, as used herein, refers to solutions of compounds that are known to be safe in formulations for pharmaceutical use and that have the effect of maintaining or controlling the pH of the formulation in the pH range desired for said formulation. Acceptable buffers for controlling pH at a moderately acidic pH to a moderately basic pH include, but are not limited to, phosphate, acetate, citrate, arginine, histidine buffers, TRIS (2-amino-2-hydroxymethyl-1,3,-propanediol) and any pharmacologically acceptable salt thereof.
The term “surfactant”, as used herein, refers to a soluble compound that can be used notably to increase the water solubility of hydrophobic, oily substances or otherwise increase the miscibility of two substances with different hydrophobicity. Surfactants are commonly used in formulations, notably in order to modify the absorption of the drug or its delivery to the target tissues. Well known surfactants include polysorbates (polyoxyethylene derivatives; Tween) as well as poloxamers (i.e. copolymers based on ethylene oxide and propylene oxide, also known as Pluronics®).
The term “stabilizing agent” or “stabilizer”, as used herein, is a compound that is physiologically tolerated and imparts a suitable stability/tonicity to a formulation. During freeze-drying (lyophilization) process or spray drying process, the stabilizer is also effective as a protectant. Compounds such as glycerine, are commonly used for such purposes. Other suitable stabilizing agents include, but are not limited to, amino acids or proteins (e.g. glycine or albumin), salts (e.g. sodium chloride), and sugars (e.g. dextrose, mannitol, sucrose, trehalose and lactose), as well as those described in the frame of the present disclosure.

The term “polymeric material” refers to polymeric components able to support high temperatures during hot melt extrusion (HME) and 3D printing. Therefore, the preferred polymeric materials according to the invention are thermoplastic polymers or thermoresistant polymers. Examples of such thermoplastic polymers that are commonly used for 3D printing are for instance are Polyvinylpyrrolidone (PVP), acrylonitrile butadiene styrene (ABS), the poly(lactic acid) (PLA), Poly(lactic-co-glycolic acid) (PLGA), the polyvinyl alcohol (PVA), poly(ε-caprolactone) (PCL), ethylene vinyl acetate (EVA). Preferably they are biodegradable or bioeliminable for more convenience to the patients. Other thermoresistant polymeric material are for instance hydroxypropyl cellulose (HPC), Hydroxypropyl methylcellulose (HPMC), Poly(Ethylene Glycol) (PEG), Eudragit derivatives (E, RS, RL, EPO), Polyvinyl caprolactam-polyvinyl acetate-polyethylene glycol graft co-polymer (Soluplus®), thermoplastic polyurethane (TPU). Suitable polymeric materials are also herein described.
The term “plasticizer” refers to a compound that can be combined with a thermoplastic polymer for instance in order to increase its plasticity or to decrease its viscosity. It can also help to decrease the glass transition temperature (Tg) of said polymer. Examples of such plasticizers that can be used in the pharmaceutical industry are for instance bio-based plasticizers such as Alkyl citrates (e.g., Acetyl triethyl citrate (ATEC), Triethyl citrate (TEC)), triacetin (TA), Methyl ricinoleate, Epoxidized vegetable oils or yet Poly Ethylene Glycol (PEG)(depending on its molecular weight, PEG can act either as polymeric matrix or as a plasticizer), castor oil, Vitamin E TPGS (D-α-tocopheryl polyethylene glycol 1000 succinate), Fatty acid esters (butyl stearate, glycerol monostearate, stearyl alcohol), pressurized carbon dioxide, surfactant (polysorbate 80) (see e.g. Crowley 2007) Suitable plasticizers are also herein described.
The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old-World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)₂, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015/197772 for instance). Antibody fragments as defined above are known in the art.
Unless otherwise specified, a value percent (%) refers to percent by weight (alternatively named wt % or % w/w.

### DETAILED DESCRIPTION OF THE INVENTION

Based on advantages of Hot Melt Extrusion (HME) and/or Fused Deposition Modelling (FDM) technologies, the inventors have developed antibody-loaded filaments that can then be used to obtain implantable devices, such as via 3D-printing using FDM technology. The present invention is based on the surprising finding that it has been possible to produce filaments comprising an antibody, said filaments having a high antibody load (at 15% and higher). The filaments could then be used to obtain implantable drug delivery devices (obtained by moulding or 3D printing for instance), from which the antibody was released in a control manner over time. Further, not only the antibody was released in a timely manner, but it was still able to bind its target. It was necessary to judiciously select the type of thermoplastic polymer to be used and to optimize the manufacturing parameters of HME, or of both HME and FDM, to obtain first a filament and then an implantable drug delivery device which could allow the stability and the affinity of the loaded antibody to be maintained.

The main object of the present invention is a filament for preparing an implantable drug delivery device, wherein the filament comprises or consists of at least one polymeric material, a plasticizer, and an active ingredient, wherein said active ingredient is an antibody. The filament may further comprise at least one stabilizer, a buffering agent and/or a surfactant. In such a case, and as an example, the filament according to the invention as a whole, can comprise or consist of at least one polymeric material, a plasticizer, an antibody and at least one stabilizer. As a further example, the filament according to the invention may comprise or consist of at least one polymeric material, a plasticizer, an antibody, at least one stabilizer and a buffering agent. The filament can be moulded or used in a 3D printer in order to obtain an implantable drug delivery device of any desired shape.

The invention further provides an implantable drug delivery device comprising or consisting of one or more layer(s) made from a filament comprising or consisting of at least one polymeric material, a plasticizer and an active ingredient, wherein said active ingredient is an antibody and wherein said filament may further comprise at least one stabilizer, a buffering agent and/or a surfactant.

A further object of the present invention is a 3D printed implantable drug delivery device obtained by 3D printing a filament comprising or consisting of at least one polymeric material, a plasticizer and an active ingredient, wherein said active ingredient is an antibody. Said filament can further comprise at least one stabilizer, a buffering agent and/or a surfactant.

Before being added to the polymeric material to form the filament and then the implantable drug delivery device, the active ingredient has to be spray-dried or freeze-dried. To do so, a preliminary liquid formulation is prepared wherein said formulation comprises or consists of the active ingredient, wherein said active ingredient is an antibody. Said liquid formulation may further comprise at least one stabilizer, a buffering agent and/or a surfactant. The liquid formulation is then spray-dried or freeze-dried according to standard methods to obtain dry microparticles. Once in the form of dried microparticles, the active ingredient is homogeneously dispersed into the at least one polymeric matrix and the plasticizer. They form an active ingredient-loaded solid dispersion such as an antibody-loaded solid dispersion.

Therefore, herein also provided is a process for producing a filament according to the invention, the process comprising the steps of:
a. preparing a liquid formulation comprising or consisting of the active ingredient, wherein said liquid formulation may further comprise at least one stabilizer, a buffering agent and/or a surfactant, and wherein said active ingredient is an antibody,
b. freeze-drying or spray-drying the liquid formulation of step a. to obtain dry microparticles,
c. dispersing homogeneously the dry microparticles of step b. with a plasticizer and at least one polymeric material (also named herein active ingredient-loaded solid dispersion),
d. extruding the dispersion of step c. by hot melting extrusion (HME) to obtain the filament.

The filament according to this invention can be used for producing an implantable drug delivery device. Said device can be either cut to a desired length, pelletized, moulded or 3D printed. The advantage of using a 3D printer is to enable the design and manufacture of novel and customized implantable drug delivery device that are not possible using traditional processes. Thanks to 3DP technology, the structure, shape or composition of the device can be customized and adapted to the patient on a case by case basis. Another advantage of using a 3D printer is to provide devices on demand.

3D printing is part of a technology called additive layer manufacturing (ALM). ALM can be based on liquid solidification or on solid material extrusion. Liquid solidification technologies include for instance Drop-on-powder deposition (DoP, or binder jetting), drop-on-drop deposition (DOD), whereas solid material extrusion technologies includes Pressure-assisted micro syringe (PAM) deposition, or yet Fused Filament Fabrication (FFF), also known as Fused Deposition Modelling™ (FDM®) technology. In a DoP or DoD system, two-dimensional layers are repeatedly printed until a three-dimensional object is formed. For example, inkjet or polyjet printing of dosage forms as disclosed herein can use additive manufacturing. The PAM technology involves the deposition of soft material (semi-solid or viscous) through a syringe-based print head. The syringe is typically loaded with the material which is then extruded using pneumatic pressure, plunger or a screw. The FDM technology is based on the extrusion of thermoplastic polymer which is driven by a gear system through a heated nozzle tip. The print head is composed of the pinch roller mechanism, a liquefier block, a nozzle and a gantry system that manages the x-y directions. The filament is fed and melt in the liquefier, turning the solid into a softened state. The solid part of the filament is used as a plunger to push the melt through the nozzle tip (Sadia et al., 2016). Once a layer of thermoplastic melt is deposited, the build platform is lowered, and the process is repeated to build the structure in a layer-wise manner.

Also encompassed by the invention is a process for producing an implantable drug delivery device, and in particular a 3D printed implantable drug delivery device, wherein the process comprises the steps of:
a. loading a filament as herein described into the print head of the 3D printer using a temperature above the glass transition temperature,
b. heating the build platform at a temperature below the glass transition temperature of the polymeric matrix;
c. depositing said heated filament through a nozzle to build the device from at least the first layer to the final top layer.

In the context of the invention as a whole, the active ingredient is an antibody. Said antibody can be any antibody as defined in the above definitions section. The antibody is preferably present in the preliminary liquid formulation, before drying, at a concentration of or of about 50 mg/ml to or to about 300 mg/mL, preferably of or of about 65 mg/ml to or to about 250 mg/mL, even preferably of or of about 80 mg/mL to or to about 200 mg/ml such as 80, 85, 90, 95, 100, 105, 110, 115, 120, 125, 130, 135, 140, 145, 150, 155, 160, 165, 170, 175, 180, 185, 190, 195 or 200 mg/mL. Alternatively, the antibody is present in the preliminary liquid formulation, before drying, at a concentration of or of about 5 to or to about 30% w/v, or preferably at a concentration of or of about 6.5 to or to about 25% w/v, even preferably of or of about 8 to about 20% such as 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20% w/v. The antibody loading in the filament, and thus in the final implantable drug delivery device, is preferably in an amount of about 15 to 40% (w/w), or in an amount of about 15 to 35% (w/w), such as 15, 16, 17, 18, 19, 20, 21, 22, 23, 24, 25, 26, 27, 28, 29, 30, 31, 32, 33, 34 or 35% (w/w).

Should at least one stabilizer be used in the context of the present invention as a whole, it is preferably a disaccharide (such as sucrose or trehalose), a cyclic oligosaccharide (such as hydroxypropyl-β-cyclodextrin), a polysaccharide (such as inulin), a polyol (such as sorbitol), or an amino acid (such as L-arginine, L-leucine, L-phenylalanine or L-proline) or any combinations thereof. Should more than one stabilizer be used, the combinations of stabilizers can be for instance (without any limitation) one disaccharide with one amino acid or a polyol with an amino acid. As an example, a combination of two stabilizers can be used, wherein one stabilizer is either sucrose or trehalose and the other stabilizer is L-arginine, L-leucine, L-phenylalanine or L-proline. The at least one stabilizer is preferably present in the preliminary liquid formulation, before drying, at a concentration of or of about 10 mg/mL to or to about 100 mg/mL, preferably of or of about 20 mg/ml to or to about 75 mg/mL, or even preferably of or of about 30 mg/mL to or to about 50 mg/ml such as 30, 31, 32, 33, 34, 35, 36, 37, 38, 39, 40, 41, 42, 43, 44, 45, 46, 47, 48, 49 and 50 mg/mL. Alternatively, the stabilizer is present in the preliminary liquid formulation, before drying, at a concentration of or of about 1 to or to about 10% w/v, or preferably at a concentration of or of about 2 to or to about 7.5% w/v, or even preferably of or of about 3 to or to about 5% such as 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% w/v.

According to the present invention in its entirety, when at least one stabilizer is present, the ratio (w/w) antibody: stabilizer(s) (alternatively referred to ratio (w/w) antibody: at least one stabilizer) in the filament, and in the implantable drug delivery device, is preferably between about 1:1 and about 5:1 (weight/weight, i.e. w/w), more preferably between about 1.2:1 and about 4:1, even more preferably between about 1.25:1 to 3:1, such as 1.25:1, 1.5:1, 1.75:1, 2.0:1, 2.25:1 and 2.5:1 (w/w). According to the present invention in its entirety, should a buffering agent be present, said buffering agent can be selected from the group comprising or consisting of (but not limited to) phosphate, acetate, citrate, arginine, trisaminomethane (TRIS), and histidine. Said buffering agent is preferably present in the preliminary liquid formulation, before drying, in an amount of from about 5 mM to about 100 mM of the buffering agent, and even preferably from about 10 mM to about 50 mM, such as about 10, 15, 20, 25, 30, 35, 40, 45 or 50 mM.

In the context of the whole disclosure, a surfactant may also be present. Said surfactant can be for instance (but without being limited to) Polysorbate 20 (PS20) or Polysorbate 80 (PS80). When present, the surfactant is preferably added in the preliminary liquid formulation, i.e. before the drying step. Said surfactant is preferably present in the preliminary liquid formulation, before drying, in an amount of or of about 0.01 to or to about 5 mg/mL, more preferably of or of about 0.01 to or to about 1 mg/mL, more particularly of or of about 0.1 to or to about 0.6 mg/ml, such as 0.1, 0.15, 0.2, 0.25, 0.3, 0.35, 0.4, 0.45, 0.5, 0.55 or 0.6 mg/mL. Alternatively, the polysorbate surfactant is preferably present in the preliminary liquid formulation, before drying, in an amount expressed in term of % weight per 100 mL (% w/v). In such a case, the polysorbate surfactant comprised in the formulations according to the present invention as a whole can be present in an amount of 0.001 to 0.5% w/v, preferably from 0.01 to 0.1% w/v, or even preferably from 0.01 to 0.06% w/v such as 0.01, 0.015, 0.02, 0.025, 0.03, 0.035, 0.04, 0.045, 0.05, 0.055 or 0.06% w/v.

In the context of the present invention, and in particular when referring to filaments or final implantable drug delivery devices, the optional at least one stabilizer, buffering agent and surfactant are regrouped under the collective name of excipients. When present, the excipients are preferably present in the filament, and thus in the final implantable drug delivery device, in a total amount of or of about 3 to or to about 20% w/w, preferably in a total amount of or of about 5 to 15% w/w, such as about 5, 5.5, 6, 6.5, 7, 7.5, 8, 8.5, 9, 9.5, 10, 10.5, 11, 11.5, 12, 12.5, 13, 13.5, 14, 14.5 or 15 wt %.

In the context of the invention as a whole, the at least one polymeric material is preferably a biodegradable, biocompatible and/or bioeliminable thermoplastic polymer such as polyurethane (TPU), polyvinylpyrrolidone (PVP), polyvinyl alcohol (PVA), poly(ε-caprolactone) (PCL), poly(lactic acid) (PLA), polydioxanone, polyglycolide, polytrimethylene carbonate, hydroxypropyl cellulose (HPC), Hydroxypropyl methylcellulose (HPMC) or combinations thereof such as, but not limited to, ethylene vinyl acetate (EVA), poly(lactic-co-glycolic acid) (PLGA), poly(L-lactide-co-caprolactone-co-glycolide)(PLGA-PCL). Polymeric materials can have a controlled size of about 200Da to about 50 kDa, preferably about 500 Da to about 40 kDa even preferably about 1 kDa to about 20 kDa, such as about 1, 2, 5, 10, 15 or 20 kDa. Alternatively, instead of having a given size (±), the polymeric materials can be a mix of polymers of different sizes, e.g. 5 kDa to 20 kDa or 7 kDa to 17 kDa. For instance, some commercially available polymers are a mix of polymers of different sizes such as Resomer® RG502 having a mix of polymers ranged between 7 and 17 kDa. Preferably said polymeric material is present in the filament, and thus in the final implantable drug delivery device, in an amount of about 50 to 75% (w/w), or in an amount of about 55 to 70% (w/w), such as 55, 56, 57, 58, 59, 60, 61, 62, 63, 64, 65, 66, 67, 68, 69 or 70%.

In the context of the invention as a whole, the plasticizer is preferably polyethylene glycol (PEG) or a PEG compound such as, but not limited to, maleimido monomethoxy PEG, activated PEG polypropylene glycol, methoxypoly(ethyleneglycol) polymer. PEG compounds according to the invention can also be charged or neutral polymers of the following types: dextran, colominic acids, or other carbohydrate-based polymers, polymers of amino acids, and biotin and other affinity reagent derivatives. PEG or PEG compounds in the context of the invention can be linear or branched. PEG or PEG compounds in the context of the invention can have a size of about 200Da to about 50 kDa, preferably about 500 Da to about 40 kDa even preferably about 1 kDa to about 20 kDa, such as about 1, 2, 5, 10, 15 or 20 kDa. Preferably said plasticizer is present in the filament, and thus in the final implantable drug delivery device, in an amount of about 2-20% (w/w), or preferably in an amount of about 5 to 15% (w/w), such as 5, 6, 7, 8, 9, 10, 11, 12, 13, 14 or 15% (w/w).

It is understood that in any case the sum of the percentages of all the components of the filaments, and thus in the final implantable drug delivery device, reaches 100%.

In the context of the whole disclosure, the implantable drug delivery device is printed using a layer thickness from about 50 μm to about 500 μm, preferably from about 100 μm to about 400 μm such as 100, 125, 150, 175, 200, 225, 250, 275, 300, 325, 350, 375 or 400 μm. The implantable drug delivery device can be designed with an infill from 0 (hollow object) to 100% (full solid object). In an embodiment, the implantable drug delivery device comprises at least one internal hollow cavity. In an alternative embodiment, implantable drug delivery the device is a fully solid object.

In a further embodiment, the present invention relates to a process for producing an implantable drug delivery device according to the invention, the process comprising:
i. cutting the filament as herein described at the appropriated length;
ii. moulding the filament as herein described until the delivery device as the appropriated form;
iii. pelletizing the filament as herein described until the delivery device as the appropriated form; or
iv. Grinding the filament as herein described to obtain a powder with a suitable particle size distribution. If needed, this powder can be future coated to modify its wettability and better control the release rate of the active ingredient. The resulting powder can be also compressed or introduced in a classical drug formulation, such as a capsule.

A non-limiting exemplary filament according to the invention comprises about 15.5% w/w of an antibody (such as a full-length monoclonal antibody or a molecule comprising a Fab fragment), about 7.5% w/w of excipients, about 69.5% w/w of a polymeric material (such as RG502), about 7.5% w/w of plasticizer (such as PEG), wherein the excipients comprise or consist of histidine (used as a buffering agent in the initial liquid formulation) and one disaccharide (either sucrose or trehalose) as the stabilizer. Another non-limiting exemplary filament according to the invention comprises about 15.5% w/w of an antibody (such as a full-length monoclonal antibody or a molecule comprising a Fab fragment), about 7.5% w/w of excipients, about 69.5% w/w of a polymeric material (RG502), about 7.5% w/w of plasticizer (PEG), wherein the excipients comprise or consist of histidine (used as a buffer in the initial liquid formulation), one disaccharide (either sucrose or trehalose) and one amino acid (L-Leucine) both acting as stabilizers.

Preferably the filaments or devices of the invention retain at least 60% of the antibody biological activity at the time of formulation and/or packaging over a period of several weeks after implantation in the subject to be treated. The activity may be measured as described in the following section “Examples” or by any other standard techniques, preferably during preliminary experiments.

The invention also provides an article of manufacture, for pharmaceutical or veterinary use, comprising a container comprising any of the above described filament or implantable drug delivery device. Also described, a packaging material providing instructions for use.

The filaments or the implantable drug delivery devices of the invention may be stored before use for at least about 12 months to about 24 months. Under preferred storage conditions, before the first use, the formulations are kept away from bright light (preferably in the dark), at temperature from about 2 to 18° C., e.g. 18° C., 15° C. or at 2-8° C. The skilled person would understand that depending on the Tg of the polymer, the temperature of storage may be higher than 18° C., such as up to 25° C. (e.g. 20° C., 22° C. or 25° C.).

The present invention provides filaments and implantable drug delivery devices, for single use, suitable for pharmaceutical or veterinary use.

### DESCRIPTION OF THE FIGURES

FIG. 1: Process to obtain filaments and 3DP devices from preliminary liquid composition (BE) and spray-dried (SD) compositions.

FIG. 2: Comparison of the HMWS levels for mAb1 formulation (mAb: stabilizer ratio 2.0:1) containing sucrose (SUC), trehalose (TRE), hydroxypropyl-beta-cyclodextrin (HP-β-CD), sorbitol (SOR) and inulin (INU) after buffer exchange (BE), spray-drying (SD) and hot melt extrusion (HME).

FIG. 3: Comparison of the HMWS levels for mAb1 formulation (mAb: stabilizer ratio 2.0:1) containing sucrose (SUC), trehalose (TRE), sucrose-leucine association (SUC-LEU) and trehalose-leucine association (TRE-LEU) after buffer exchange (BE), spray-drying (SD), hot melt extrusion (HME) and 3D printing (3DP).

FIG. 4: (a) Dissolution profiles of 3DP device containing mAb1 stabilized with TRE-LEU (3DP_7; solid line) and in vitro pH values variation of the surroundings medium over dissolution time was showed in the dissolution chart (dashed line). (b) Degradation of the PLGA contained into the 3DP device over 10 weeks in the dissolution medium at 37° C.

FIG. 5: Comparison of monomer, HMWS and LMWS levels (%) of mAb1 released from 3DP_7 during the in vitro dissolution test. The mAb1 reference was characterized with 97.4±0.4% (monomer), 2.6±0.4% (HMWS) and no LMWS.

FIG. 6: Comparison of the binding capacity of mAb1 released from 3DP_7 after 24 h, 5, 10 and 15 weeks of dissolution.

FIG. 7: In vitro release profiles of 3DP devices containing mAb1 stabilized with TRE-LEU association (3DP_42 (10% infill), 3DP_43 (50% infill), 3DP_44 (100% infill)). Devices were printed with a layer thickness of 0.3 mm.

FIG. 8: Comparison of the HMWS levels for fAb2 formulation (Fab: stabilizer ratio 2.0:1) containing SUC, SUC-LEU, TRE and TRE-LEU after SD, HME and 3DP. The fAb2 reference was characterized with a monomer content and a HMWS level of 99.6±0.2% and 0.4±0.2%, respectively.

FIG. 9: Dissolution profiles of 3DP DDS containing fAb2 stabilized with SUC (3DP_F1), SUC-LEU (3DP_F2), TRE (3DP_F3) and TRE-LEU formulation (3DP_F4).

FIG. 10: Comparison of the monomer content (a) and HMWS level (b) of fAb2 released from 3DP_F1, 3DP_F2, 3DP_F3 and 3DP_F4 over time (8 weeks).

FIG. 11: Binding capacity of fAb2 released from 3DP_F1, 3DP_F2, 3DP_F3 and 3DP_F4 after 24 h of dissolution.

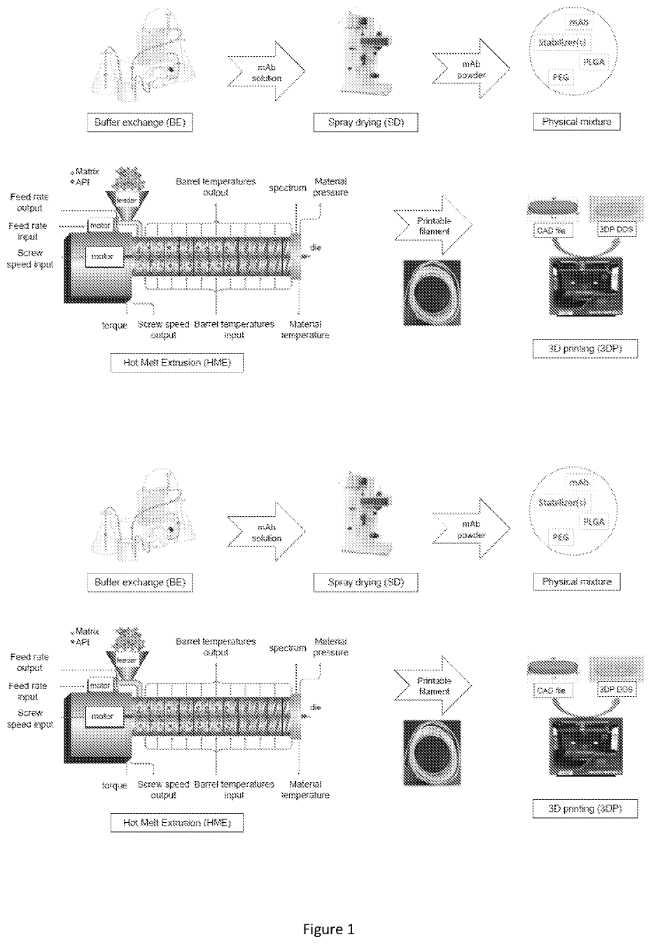

### EXAMPLES

### Abbreviations

HMWS=high molecular weight species; LMWS=low molecular weight species; SD=spray-drying or spray-dried; HME=Hot melt extrusion; 3DP=Three-dimensional printing or Three-dimensional printed; BE: Buffer exchange; DDS: Drug delivery system; DDD: Drug delivery device; DSC: Differential scanning calorimetry; FDM: Fused deposition modelling; HME: Hot melt extrusion; LEU: L-leucine; Mw: Molecular weight; mAb: full length monoclonal antibody; fAb: Fab fragment of an antibody; PBS: Phosphate buffer solution; gel permeation chromatography (GPC); SEC=Size exclusion chromatography; PEG: Polyethylene glycol; PLGA: Poly(lactide-co-glycolide) acid; rpm: Revolutions per minute; SUC: Sucrose; Tg: Glass transition temperature; TGA: Thermogravimetric analysis; Tm: Melting temperature; TRE: Trehalose; % (w/w): Weight percentage; Stab: stabilizer; HP-β-CD: Hydroxypropyl-β-cyclodextrin; SOR: sorbitol; INU: inulin.

### 1. Materials

mAb1 is an IgG4, has a molecular weight (MW) of about 150 kDa and a pl of about 6.0-6.3.

fAb2 is a Fab moiety of an antibody. fAb2 has a MW of about 50 kDa and a pl of about 9.3-9.6.

### 2. Methods

### 2.1. Spray-Drying

The antibody-containing solutions were spray-dried using a lab-scale Spray-Dryer B-290 (Büchi Labertechnik) equipped with a 0.7 mm nozzle. Settings were based on standard procedure and kept constant for all formulations. The solutions to be spray-dried were preliminary prepared in a 15 mM histidine buffer at pH 5.6, with other excipients on demand. An overview of the mAb1 and fAb2 solution compositions, concentrations and mAb: stabilizer ratios are shown in Tables 1 & 8. All powders were sealed in a polypropylene container and stored in a desiccator under vacuum.

### 2.2. Hot Melt Extrusion

Printable filaments were prepared from physical mixtures of raw PLGA, PEG 2 kDa and mAb1- or fAb2-containing spray-dried (SD) powders which were previously blended together using a Turbula® mixer (Willy A. Bachofen AG). The blend was manually fed into a 11-mm twin screw extruder (Process-11, Thermo Fischer Scientific), equipped with modular screws (L/D-ratio 40:1), and a round die with a diameter of 1.6 mm. The barrel was heated using a gradient of temperature controlled by eight thermocouples. The feeding zone was maintained at room temperature using a water circulator. The three first segments were set at 20, 40 and 80° C., respectively. The middle segments, from the 4th to 6th thermocouple, were set at 90° C. The last thermocouple, which was located right before the die, was set at 85° C. and the die itself was set at 75° C. For all experiments, the screw speed was set at 40 rpm during the feeding and 60 rpm when the filament was manually coiled. These parameters were kept constant (see Table 1).

### 2.3. 3D Printing of Antibody-Loaded Devices

The design of the devices was drawn using the 3D computer-aided design (CAD) software ThinkerCAD™ (AutoDesk® Inc.) and exported into a software for slicing. The dimensions of the devices were 20×5×2 mm (length, width, height) for a volume of 178.43 mm³. An Hyrel 3D system 30M printer (GA), equipped with a 0.5 mm MK2-250 hot extruder, was used to print the mAb1- and fAb2-loaded devices. The temperature of the build platform did not need to be controlled. The printing temperature was set at 105±2° C. The printing speed was set at 1 mm/s for the first layer and 10 mm/s for the others. The layer thickness of the devices was set at 0.1 mm and 0.3 mm to evaluate its influence on the potential degradation of the loaded mAb1 as well as on its release profile. The printing of devices was performed with an infill of 100% (v/v), except specified otherwise in the examples below.

### 2.4. Analytical Methods

Differential scanning calorimetry (DSC): Thermal analyses of SD powders, filaments, 3DP DDS were performed, according to a standard method, via DSC using a heat-flux type DSC Q2000 (TA instruments) equipped with a cooling system.

Thermogravimetric analysis (TGA): TGA were performed on a Q500 TGA (TA instrument), equipped with a balance with a sensitivity of 0.1 μg, as per standard methods. Data collection and analysis were performed using TA Instruments® Trios 4.5.0 software.

Molecular weight analysis of polyesters by size exclusion chromatography (SEC) in chloroform: Number average molecular weight (Mn), weight average molecular weight (Mw), and polydispersity index (Mw/Mn) of polyesters were measured by SEC, as per standard methods. Relative molecular weights (number and weight average) and polydispersity index were calculated by reference to a polystyrene standard calibration curve established using the same experimental conditions. Means and standard deviations (STD) related to molecular weights and polydispersity were calculated as detailed above for NMR analysis.

Antibodies stability evaluation: The quantification of mAb1 monomer as well as the evaluation of both HMWS and LMWS contents was carried out by size exclusion high performance liquid chromatography. This analysis was conducted on samples obtained from either dissolution studies or after extraction from the printable filaments and 3DP devices. These quantifications were performed on an Agilent 1200 series LC system equipped with a UV detector (Agilent Technologies), according to standard protocols. The mobile phase was a 0.2 M PBS solution, at pH 7.0. The calibration curve of mAb1 was ranged from 20 to 2000 μg/mL. The stability of mAb1 was evaluated using the percentage of monomer loss, which corresponded to the difference in the percentage of monomers before and after both HME and 3DP processes. Monomer, HMWS and LMWS levels (%) were compared to a reference that consisted of mAb1 solution obtained after buffer exchange. Similar method was used for fAb2 stability evaluation.

Antibodies extraction from the polymeric matrix: To evaluate the stability of mAb1 that was melt-encapsulated in both printable filaments and 3D-printed devices, samples of approximatively 10 mg were placed in Nanosep® with 0.2 μm Bio-Inert centrifugal devices (Pall) and dissolved in 0.5 mL of dichloromethane. The Nanosep® devices were stirred at 600 rpm during 2 hours at room temperature to dissolve the PLGA, using a Thermomixer confort® tubes mixer (Eppendorf AG). The samples were centrifuged at 12 000 rpm during 10 min and the medium was withdrawn. Then, 0.5 mL of dichloromethane were added again. The sample was stirred for 5 minutes and centrifuged as previously mentioned. This step was repeated twice. Dichloromethane was removed and the Nanosep® devices containing the mAb's precipitate were placed 1 hour under vacuum to remove potential residual solvent. Then, 0.5 mL of PBS (0.2 M, pH 7.0), containing 0.02% w/w of polysorbate 80 (PS80), were added in the tube to solubilize mAb1 before being stirred at 600 rpm for 2 hours. Then, the Nanosep® devices were centrifuged 10 min at 12 000 rpm (Adapted from Arrighi et al., 2019). mAb1 stability was evaluated by SEC (as described above). Similar method was used for fAb2 extraction.

Antibodies loading after melt-encapsulation: The amount of encapsulated mAb1 into PLGA matrix was determined using colorimetric detection by bicinchoninic acid (BCA) protein assay according to standard methods. The Pierce™ microplate procedure was carried out to determine the amount of melt-encapsulated mAb1. The quantification of both standards and samples was performed at 562 nm on a SpectraMax M5 microplate reader (Molecular Devices) at room temperature. Overall, mAb1 loading was determined as follows:

mAb loading (%)=(amount of melt-encapsulated mAb)/(amount of 3DP device)*100.

Similar method was used for fAb2 loading.

Dissolution studies: To evaluate the release profiles of the loaded mAb1/fAb2 from 3DP DDS, in vitro dissolution studies were performed. 3DP devices (˜200 mg) were placed in 5 mL Eppendorf® tube filled with 5 mL of PBS (0.2 M, pH 7.0, 37° C.) and stirred at 600 rpm using a Thermomixer confort® tubes mixer (Eppendorf AG) (adapted from (Marquette et al., 2014)). At predetermined times, 5 mL of medium was withdrawn, collected and filtrated on 0.45 μm PVDF Acrodisc® syringe filters (Pall). Similar volume was replaced with fresh buffer (5 mL). The filtrated solutions were measured using SEC analysis equipped with UV-detector at 280 nm and analysed for pH.

PLGA degradation during dissolution: The decrease in polymer molecular weight (Mw) of PLGA during the drug release was carried out using gel permeation chromatography (GPC). The protocol was similar to that used for the dissolution test. Mw were calculated using polystyrene standards.

Enzyme-linked immunosorbent assay (ELISA test): The binding capacity of the mAb1/fAb2 was assessed using an ELISA test, according to standard methods.

Data analysis: All experiments were performed in triplicate, unless otherwise specified. Prism 8 software (GraphPad software) was used for statistical analysis. The results are expressed as a mean±standard deviation. Statistical significance was determined at p-value<0.05 using ANOVA and Turkey's or Dunnett's post-hoc test (as recommended by Prism software).

### Example 1—Preparation of the Printable Filaments and 3DP DDS Loaded With mAb1

The mAb1 solutions were formulated with different stabilizers (see Table 1). These liquid solutions were spray-dried to produce mAb1-loaded powders. Indeed, mAb1 was used in solid state to increase its stability and to facilitate the handling during further processing. Then, a mixture of mAb1-loaded powder, Resomer® RG502 as a polymeric material (Evonik Industries) and PEG as a plasticizer was extruded using HME to produce filaments suitable for printing. These printable filaments were used to feed the 3DP printer to print the devices (alternatively herein named drug delivery device or implantable drug delivery device). Optimal formulations were identified by evaluating mAb1 integrity after each manufacturing step (SD, HME, 3DP). Finally, in vitro evaluations (dissolution test and binding capacity) were performed.

### Example 2—Preliminary Study on Raw Materials and Printable Filaments With mAb1

The thermal properties, including their temperature of degradation, of all raw materials were assessed using TGA and DSC analysis, respectively.

The degradation temperature of raw RG502 was around 175° C. No apparent weight loss was observed under 200° C. on raw PEG and on the extruded filaments loaded with mAb1. No residual moisture was observed in RG502 and PEG raw materials. These results confirmed that all raw materials seemed stable and may be processed according to the temperatures in both HME and 3DP (90° C. and 105° C., respectively). Indeed, only the mass loss was characterized using TGA and other methods were required to state on mAb1 stability such as SEC and binding capacity.

The TGA thermograms of the SD mAb1 powder showed a slight weight loss (˜4% w/w) when a temperature of 100° C. was reached. Such decrease could be attributed to the residual moisture content into the SD mAb1 powder (about 3.4±0.8%). A second weight loss was observed above 150° C. on all the SD mAb1-loaded powders. The mAb1-loaded powders were thus able to ensure the stability of mAb1 during both HME and 3DP.

Then, DSC analyses were carried out to evaluate the influence of the addition of PEG and mAb1-loaded SD powder on the T_{g} of the thermoplastic polymer RG502. Indeed, the aim of this work being to develop mAb1-loaded 3DP DDS, the T_{g} should be as low as possible to allow decreasing the temperature of the different processes (HME, 3DP) and the potential degradation of the biotherapeutic as a consequence.

TABLE 1
Composition of evaluated mAb1 formulations: liquid composition after buffer exchange and
before spray-drying (SD) (expressed in % w/v), solid composition of spray dried powders (expressed
in % w/w), printable filaments produced using hot melt extrusion (HME) batches (expressed in % w/w)
and associated 3D printing (3DP) batches with layer thickness of 0.1 mm and 0.3 mm. mAb1 was at
8% w/v in the initial liquid composition

| | | | HIS | Stab. | LEU | HIS | Stab. | LEU | mAb1 | HME | RG502 | PEG | Excipient | mAb1 | 3DP | 3DP |
|---|---|---|---|---|---|---|---|---|---|---|---|---|---|---|---|---|
| SD | | mAb: | liquid composition | | | Solid composition | | | | batch | (% | (% | (% | (% | 0.1 mm | 0.3 mm |
| batch | | stab. | (% w/v) after | | | (% w/w) | | | | No. | w/w) | w/w) | w/w) | w/w) | batch | batch |
| No. | Stab. | ratio | buffer exchange | | | after SD | | | | Filament after HME | | | | | No. | No. |
| SD_1 | SUC | 1.5:1 | 0.2 | 5.1 | — | 1.7 | 38.3 | — | 60.0 | HME_1 | 60.1 | 6.6 | 13.3 | 20.0 | — | — |
| SD_2 | | 2.0:1 | 0.2 | 3.8 | — | 1.9 | 31.4 | — | 66.7 | HME_2 | 62.9 | 6.9 | 10.1 | 20.1 | — | — |
| SD_3 | | 2.5:1 | 0.2 | 3.2 | — | 2.1 | 26.5 | — | 71.4 | HME_3 | 64.1 | 7.1 | 8.7 | 20.2 | — | — |
| SD_4 | TRE | 1.5:1 | 0.2 | 5.1 | — | 1.7 | 38.3 | — | 60.0 | HME_4 | 60.1 | 6.6 | 13.3 | 20.0 | — | — |
| SD_5 | | 2.0:1 | 0.2 | 3.8 | — | 1.9 | 31.4 | — | 66.7 | HME_5 | 62.9 | 6.9 | 10.1 | 20.1 | — | — |
| SD_6 | | 2.5:1 | 0.2 | 3.2 | — | 2.1 | 26.5 | — | 71.4 | HME_6 | 64.1 | 7.1 | 8.7 | 20.2 | — | — |
| SD_7 | SOR | 1.5:1 | 0.2 | 5.1 | — | 1.7 | 38.3 | — | 60.0 | HME_7 | 60.1 | 6.6 | 13.3 | 20.0 | — | — |
| SD_8 | | 2.0:1 | 0.2 | 3.8 | — | 1.9 | 31.4 | — | 66.7 | HME_8 | 62.9 | 6.9 | 10.1 | 20.1 | — | — |
| SD_9 | | 2.5:1 | 0.2 | 3.2 | — | 2.1 | 26.5 | — | 71.4 | HME_9 | 64.1 | 7.1 | 8.7 | 20.2 | — | — |
| SD_10 | INU | 1.5:1 | 0.2 | 5.1 | — | 1.7 | 38.3 | — | 60.0 | HME_10 | 60.1 | 6.6 | 13.3 | 20.0 | — | — |
| SD_11 | | 2.0:1 | 0.2 | 3.8 | — | 1.9 | 31.4 | — | 66.7 | HME_11 | 62.9 | 6.9 | 10.1 | 20.1 | — | — |
| SD_12 | | 2.5:1 | 0.2 | 3.2 | — | 2.1 | 26.5 | — | 71.4 | HME_12 | 64.1 | 7.1 | 8.7 | 20.2 | — | — |
| SD_13 | HP- | 1.5:2 | 0.2 | 5.1 | — | 1.7 | 38.3 | — | 60.0 | HME_13 | 60.1 | 6.6 | 13.3 | 20.0 | — | — |
| SD_14 | β- | 2.0:2 | 0.2 | 3.8 | — | 1.9 | 31.4 | — | 66.7 | HME_14 | 62.9 | 6.9 | 10.1 | 20.1 | — | — |
| SD_15 | CD | 2.5:2 | 0.2 | 3.2 | — | 2.1 | 26.5 | — | 71.4 | HME_15 | 64.1 | 7.1 | 8.7 | 20.2 | | |
| SD_16 | SUC | 2.0:1 | 0.2 | 3.8 | — | 1.9 | 31.4 | — | 66.7 | HME_16 | 69.4 | 7.6 | 7.6 | 15.3 | 3DP_1 | 3DP_2 |
| SD_17 | SUC | 2.0:1 | 0.2 | 3.2 | 0.6 | 1.9 | 26.4 | 5 | 66.7 | HME_17 | 69.4 | 7.6 | 7.6 | 15.3 | — | 3DP_3 |
| SD_18 | TRE | 2.0:1 | 0.2 | 3.8 | — | 1.9 | 31.4 | — | 66.7 | HME_18 | 69.4 | 7.6 | 7.6 | 15.3 | 3DP_4 | 3DP_5 |
| SD_19 | TRE | 2.0:1 | 0.2 | 3.2 | 0.6 | 1.9 | 26.4 | 5 | 66.7 | HME_19 | 69.4 | 7.6 | 7.6 | 15.3 | — | 3DP_6 |
| SD_20 | TRE | 2.0:2 | 0.2 | 3.2 | 0.6 | 1.10 | 26.5 | 6 | 66.8 | HME_20 | 69.4 | 7.6 | 7.6 | 15.3 | — | 3DP_7 |

The T_{g} of RG502 was found to be 38.0±0.7° C., which was consistent with data already described in literature (Pignatello et al., 2009). PEG was characterized by a sharp endothermic peak at 52° C. The T_{g} of RG502 decreased to 21.8±0.4° C. when PEG and SD powder were added during HME (data not shown). Such decrease of the T_{g}, in addition to the loss of the sharp melting peak of PEG, demonstrated that mAb1-loaded SD powder and PEG were properly dispersed in the molten polymeric matrix (Zhang et al., 2017).

### Example 3—Formulation Screening and mAb1 Stability After Spray-Drying Process

Stabilizers were selected to maintain antibody integrity during all the steps of manufacturing. The main expected deleterious factor was the relatively high temperatures that were used during both HME and 3DP. Unfortunately, stabilizer selection is not universal and needs to be adapted to each biotherapeutic and in regard with the stress factors associated to the process (Le Basle et al., 2020; Wang et al., 2007). SUC, TRE, HP-β-CD, SOR and INU are commonly used in formulations comprising antibodies (Baek et al., 2017; Bowen et al., 2013; Gidwani and Vyas, 2015; Kanojia et al., 2016; Maury et al., 2005). The effect of the addition of stabilizers on the stability of the loaded mAb1 was investigated using 3 different mAb: stabilizer ratios (w/w) (1.5:1, 2.0:1 and 2.5:1) (see formulations of Table 1). A mAb: stabilizer ratio (w/w) 2.0:1 was previously described to increase the stability of mAb1 during a SD process (Bowen et al., 2013). Higher and lower ratios were also investigated to evaluate their influence on the stability of our own mAb1, not only during SD, but more especially during HME and 3DP (two steps bringing a high thermal stress).

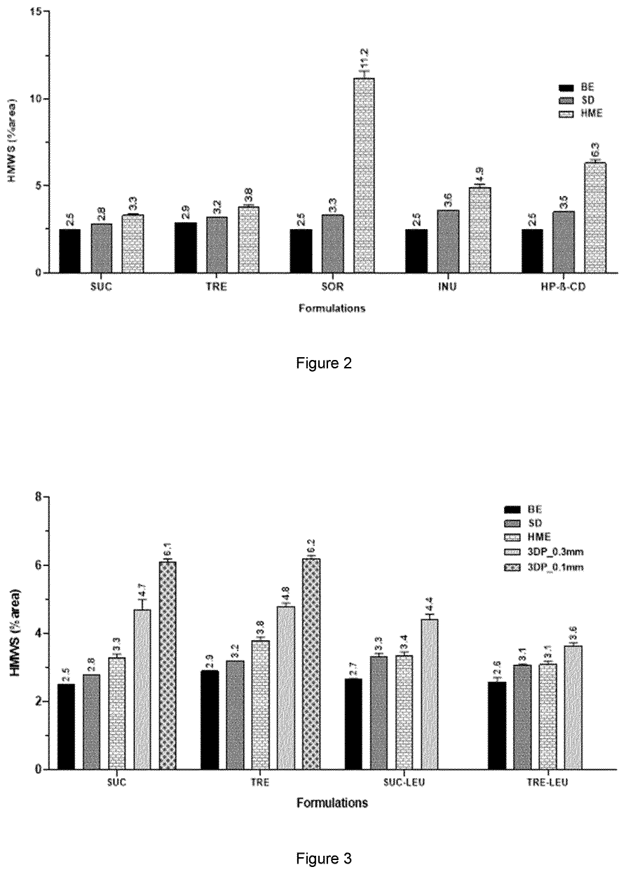

The different liquid compositions to be assessed were obtained by buffer exchange. No instabilities were observed between mAb1 reference (before buffer exchange) and the various liquid compositions (after buffer exchange, BE). The percentages of HMWS were quite similar to that observed from mAb1 reference (2.6±0.4%) (Table 2). After SD, there was no significant formation of HMWS either for mAb: stabilizer ratios of 1.5:1 and 2.0:1, regardless of the nature of the stabilizer (p-value>0.05) (FIG. 2). In contrast, when a ratio of 2.5:1 was used, the percentage of HMWS increased, regardless of the nature of the stabilizers, excepted for SUC and TRE (p-value>0.05) (Table 2). LMWS level was also assessed and no fragmentation was observed on the raw mAb1 solution. Similar observations were made after BE and SD, regardless the mAb: stabilizer ratio (Table 2). As ratios of 1.5:1 and 2.0:1 showed similar results, ratio 2.0:1, allowing a higher proportion of mAb1 versus stabilizers, was selected for further investigations.

TABLE 2
Comparison of HMWS and LMWS levels of mAb1 formulation (mAb:
stabilizer ratios: 1.5:1; 2.0:1 and 2.5:1; see Table 1 for formulations)
after the buffer exchange (BE), spray-drying (SD) and hot
melt extrusion (HME). HMWS and LMWS are all expressed in %.

| mAb: | | | | | | | | | | |
|---|---|---|---|---|---|---|---|---|---|---|
| stabilizer ratio | | 1.5:1 | | | 2.0:1 | | | 2.5:1 | | |
| Stab | Attributes | BE | SD | HME | BE | SD | HME | BE | SD | HME |
| SUC | HMWS | 2.5 | 2.7 ± 0.3 | 3.2 ± 0.2 | 2.5 | 2.8 ± 0.1 | 3.3 ± 0.3 | 2.9 | 3.51 ± 0.04 | 4.2 ± 0.2 |
| | LMWS | 0.0 | 0.0 ± 0.0 | 0.0 ± 0.0 | 0.0 | 0.0 ± 0.0 | 0.0 ± 0.0 | 0.0 | 0.0 ± 0.0 | 0.0 ± 0.0 |
| TRE | HMWS | 2.5 | 2.8 ± 0.8 | 3.2 ± 0.5 | 2.8 | 3.2 ± 0.5 | 3.8 ± 0.5 | 2.9 | 3.54 ± 0.02 | 4.3 ± 0.1 |
| | LMWS | 0.0 | 0.0 ± 0.0 | 0.0 ± 0.0 | 0.0 | 0.0 ± 0.0 | 0.0 ± 0.0 | 0.0 | 0.0 ± 0.0 | 0.0 ± 0.0 |
| SOR | HMWS | 2.3 | 3.1 ± 0.1 | 14.6 ± 0.5 | 2.5 | 3.3 ± 0.2 | 11.2 ± 0.4 | 2.4 | 3.22 ± 0.03 | 8.9 ± 0.3 |
| | LMWS | 0.0 | 0.0 ± 0.0 | 0.0 ± 0.0 | 0.0 | 0.0 ± 0.0 | 0.4 ± 0.1 | 0.0 | 0.00 ± 0.01 | 0.2 ± 0.1 |
| INU | HMWS | 2.5 | 3.7 ± 0.2 | 5.4 ± 0.2 | 2.5 | 3.6 ± 0.3 | 4.9 ± 0.1 | 2.6 | 4.05 ± 0.02 | 4.8 ± 0.1 |
| | LMWS | 0.0 | 0.0 ± 0.0 | 0.0 ± 0.0 | 0.0 | 0.0 ± 0.0 | 0.0 ± 0.0 | 0.0 | 0.0 ± 0.0 | 0.0 ± 0.0 |
| HP-β- | HMWS | 2.8 | 3.0 ± 0.3 | 9.4 ± 08 | 2.5 | 3.5 ± 0.5 | 6.3 ± 0.2 | 2.8 | 4.10 ± 0.02 | 8.0 ± 0.5 |
| CD | LMWS | 0.0 | 0.0 ± 0.0 | 0.0 ± 0.0 | 0.0 | 0.0 ± 0.0 | 0.10 ± 0.05 | 0.0 | 0.02 ± 0.01 | 0.23 ± 0.02 |
| mAb | HMWS | 2.6 ± 0.4 | | | | | | | | |
| reference | LMWS | 0.0 ± 0.0 | | | | | | | | |

### Example 4—Extrusion of mAb1-Loaded Printable Filaments

To obtain filaments, mAb1-loaded SD powders were mixed with PLGA and PEG and extruded (HME) to make printable filaments (see formulations of Table 1). The filaments were successfully prepared with a diameter between 1.70 and 1.75 mm as recommended to feed the FDM 3D printer (Melocchi et al., 2015). mAb1 loading was chosen at 15% (w/w).

As shown in Table 2 and FIG. 2, the percentage of HMWS increased, due to the use of relatively high temperature, regardless the nature of the stabilizer (p-value<0.0001). The percentage of HMWS reached 6.4±0.2%, 11.2±0.5% and 4.9±0.1% when HP-β-CD, SOR and INU respectively were added in the formulations (mAb: stabilizer ratio 2.0:1) (see FIG. 2). In contrast, SUC and TRE seemed the most adapted to stabilize mAb1 during the HME process that was performed at 90° C. Indeed, the percentages of HMWS only increased to 3.3±0.3%) and 3.8±0.5%), respectively (FIG. 2). No significant difference was highlighted for both disaccharides after the HME process (p-value>0.05).

The percentage of LMWS was also evaluated after HME (see Table 2). It was observed that slight fragmentation appeared when HP-β-CD and SOR were used as stabilizers. In contrast, no LMWS were observed with SUC, TRE and INU.

Overall, HP-β-CD, SOR and INU were less effective to maintain mAb1 stability during HME in comparison to SUC and TRE. Based on the evaluation of HMWS and LMWS levels, mAb1 integrity was ensured during HME using TRE and SUC as stabilizers.

Examples 2 and 3 have shown that SUC and TRE seemed to be the most suitable stabilizers to stabilize the formulations over the successive steps of production (after SD and HME).

Finally, mAb1 loading was assessed on the printable filaments before the printing process. This showed that the real loadings of all the filaments were similar to the targeted one (15% w/w) with very low standard deviations (Table 3). These results indicated that the manufacturing process was suitable and reproducible to produce uniform printable filaments with homogeneous dispersion.

TABLE 3
mAb1 loading in printable filaments and
3DP devices obtained by BCA assay.

| HME batch name | Loading (% w/w) | 3D batch name | Loading (% w/w) |
|---|---|---|---|
| HME_16 | 16.0 ± 0.1 | 3DP_2 | 15.8 ± 0.2 |
| HME_18 | 16.2 ± 0.1 | 3DP_5 | 16.2 ± 0.3 |

### Example 5-3D Printing of mAb1 Implantable Delivery Devices

A slicing software was used to design a model of implantable 3DP device with a shape that could be implantable. The printing process was performed in a room at 20° C. Indeed, physical state of the filaments may be quickly modified due to the room temperature as it was previously mentioned that their T_{g} was around 22° C. Therefore, at 20° C., filaments were able to be printed as their stiffness was preserved. However, the handling of the filaments induced a heat transfer by conduction. This phenomenon was greater when the filaments were loaded in the print head. Indeed, they were too soft to be travelled along the feeding gears. To limit the heat transfer by conduction during printing, 3DP had to be performed using a “flexible hot flow” modular printing head MKE-250.

The device resolution was macroscopically evaluated and, when the infill was set at 100%, a fully solid device was expected. Immediate visualization showed defects and a lack of matter at the top of the devices (data not shown). The printing step was performed at 105° C. which was the temperature where both adhesion to the build platform and between successive layers were promoted. The printing speed was selected at 1 mm/s for the first layer and 10 mm/s for the following layers to improve the resolution of DDS. 3D printings with a layer thicknesses of 0.1 mm and 0.3 mm were evaluated.

Extraction of mAb1 was performed on 3DP devices to evaluate the percentage of both HMWS and LMWS. The percentage of HMWS increased following 3DP, regardless of the layer height as well as the nature of the disaccharide (FIG. 3). However, it was significantly higher when a layer thickness of 0.1 mm was used (p-value<0.0001 and p-value<0.0004, respectively). For instance, HMWS percentage has increased from 3.3±0.1% (formulation HME_16) and 3.8±0.1% (formulation HME_18) after HME to 4.7±0.3% (formulation 3DP_2) and 4.8±0.1% (formulation 3DP_5) after 3DP with a layer thickness of 0.3 mm, or to 6.14.±0.1% (formulation 3DP_1) and 6.2±0.1% (formulation 3DP_4) after 3DP with a layer thickness of 0.1 mm, when SUC and TRE were used, respectively. This was attributed to the relatively high temperature that was used during 3DP. This may be explained by the slower movement of build platform which led to an extended area of contact between the nozzle of the printer and the printed devices (Carlier et al., 2019).

Despite the addition of SUC or TRE, it was demonstrated that a significant (although acceptable) increase of HMWS appeared after 3D printing. Therefore, it was hypothesized that the addition of a hydrophobic amino acid such as LEU (Minne et al., 2008) could enhance the stability of the loaded mAb1. 3DP devices were printed using a layer thickness of 0.3 mm, starting with preliminary liquid formulations comprising the combination of stabilizers SUC-LEU or TRE-LEU (see Table 1).

HMWS levels were evaluated after each process (from SD to 3DP, the starting values being those for BE) (see FIG. 3). After 3DP, these levels were 4.4±0.2% and 3.6±0.1% for 3DP_3 and 3DP_6, respectively. These levels were compared to those obtained when SUC and TRE were used alone. It was demonstrated that the addition of LEU to SUC and TRE was able to limit the production of HMWS. The decrease of HMWS was more significant with the association of TRE-LEU (p-value<0.0001). The rate of increase was compared as a ratio over the whole process (between 3DP and SD). Following the addition of LEU to TRE, the percentage of HMWS increased by about 18% versus 50% when TRE was formulated alone. The same trend was observed when LEU was added to SUC with higher HMWS levels (SUC-LEU: 33% vs SUC: 66% increase).

LMWS levels were also investigated after 3DP. It was demonstrated that a slight increase of LMWS (around 0.05±0.04%), regardless the addition of LEU to SUC or TRE (data not shown).

Finally, drug loadings were assessed on 3DP DDS and the BCA results showed real loadings closed to the targeted loading of 15% (w/w) (see Table 4). These results confirm the uniform dispersion of mAb1 in the polymeric matrix expressed after HME.

TABLE 4
mAb1 loading in printable filaments and
3DP devices obtained by BCA assay.

| HME batch name | Loading (% w/w) | 3D batch name | Loading (% w/w) |
|---|---|---|---|
| HME_17 | 15.2 ± 0.1 | 3DP_3 | 15.1 ± 0.2 |
| HME_19 | 15.6 ± 0.2 | 3DP_6 | 15.5 ± 0.2 |
| HME_20 | 15.9 ± 0.5 | 3DP_7 | 15.5 ± 0.5 |

It was therefore a surprising finding from the inventors that it was possible 1) to extrude printable filaments by HME starting from mAb1-loaded SD and 2) to create 3DP devices via FDM with said filaments. The increase of HMWS observed mainly after HME and 3DP was directly related to the thermal degradation occurring at 90° C. (during HME) and 105° C. (during 3DP). The most promising formulation, containing TRE-LEU and to a lesser extend SUC-LEU, and able to minimize the production of HMWS and to promote mAb1 stability, was further investigated.

### Example 6—Dissolution Tests on the 3D Printed Device Containing mAb1

It was previously demonstrated and described that PLGA-based drug delivery system (DDS; e.g. microparticles and implants) are characterized by a triphasic release profiles. It could be more interesting to promote a release profile where a limited latent phase occurred. Indeed, the latent phase could lead to mAb1 degradation due to its retention in the polymeric matrix and the medium uptake. Moreover, a linear release profile which could tend towards a “zero order kinetic” should allow a constant drug release and a steady release concentration of the mAb1 in the dissolution medium.

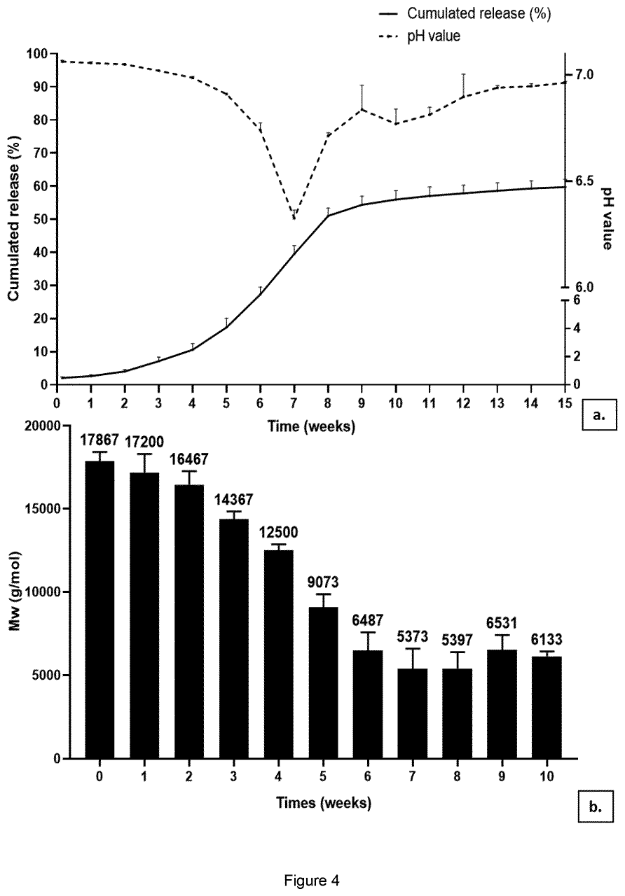

As shown in FIG. 4a, the release of mAb1 from the 3D printed device was characterized by a low burst effect 2.0±0.3% within 24 h. The sustained release occurred over time starting with a slow release phase (latent phase) within the first weeks. Weeks 1 to 4 showed indeed a low antibody release up to 10.6±1.9%. This was due to the struggle of medium to penetrate the PLGA matrix and is known to be low during the first weeks. Then, an increase of the percentage of release of mAb1 was observed in the following weeks. The cumulative release accelerated and increased from 17.3±2.8% after 5 weeks to 57.8±2.5% after 12 weeks. Finally, a low release phase was observed to reach 59.7±2.3% after 15 weeks. The release of mAb1 was dependent on the water uptake which allows mAb1 diffusion through the pores of the device.

Degradation of the polymer RG502 was evaluated on the 3D printed device during the dissolution test (FIG. 4b). The diffusion of the medium through the polymeric matrix is needed to trigger the hydrolysis and promotes the erosion of the DDS. The PLGA derivatives, Resomer® RG502, was characterized with an initial Mw of 17 867±577 g/mol. RG502 hydration occurred during the first weeks of the dissolution test. Degradation of the polymer was marginally observed, and pH value of the surrounding medium remained constant (FIG. 4a). Then, degradation increased after 3 weeks with a loss of around 20% (14 367±462 g/mol) of its initial mass (loss due to the hydrolytic scission of RG502 in oligomers into the devices). The erosion started after 3 weeks according to the decreased pH value of the surrounding medium (FIG. 4a). During the first weeks, mainly the degradation occurred but the onset of erosion was triggered and accelerated with the pH drop. Therefore, the autocatalysis accelerated the erosion and increased both PLGA degradation and mAb1 release. For instance, a loss of 64% (5373±1217 g/mol) of its initial mass was observed after 7 weeks of dissolution (FIG. 4b). Interestingly, the pH value decrease to 6.3±0.1% which demonstrated the highest rate of erosion. No further degradation was reported after this main degradation and polymer's Mw remained stable around 6000 g/mol (FIG. 4b). Moreover, the erosion rate decreased after week 7. This statement was demonstrated by the increase of the pH value in the following weeks from 6.7±0.1% (week 8) to 7.0±0.1% after 15 weeks (FIG. 4a). Such profile was in line with expectations (Cossé et al., 2016; Ghalanbor et al., 2013).

The release of mAb1 was assessed over 15 weeks. The pH values remained slightly acidic due to the generation of oligomers and their diffusion to the dissolution medium. No further degradation of the PLGA nor further release of mAb1 was observed after 10 weeks. As the sample generated after 10 weeks in the dissolution medium remained insoluble in chloroform, it is likely that PLGA and mAb1 form insoluble aggregates over time.

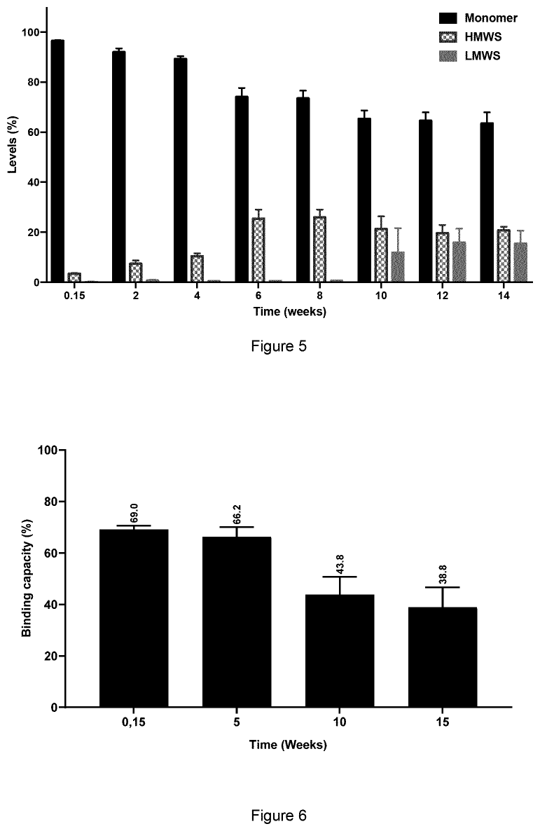

In order to study the stability of mAb1 during release, HMWS and LMWS levels as well as the monomer content were assessed in the dissolution test (FIG. 5). It was shown that a decrease of monomer percentage was associated with an increase of either HMWS or LMWS species. The highest HMWS levels were observed between week 6 (25.4±3.6%) and week 8 (25.9±3.1%). This increase was correlated with the highest erosion rate previously discussed and the decrease of the pH to 6.3±0.1 at week 7. Interestingly, a slight increase of LMWS was observed (<0.7%) during the first 9 weeks of dissolution. LMWS levels increased to 17.0±5.7% after 10 weeks. This level remained high with a value of 15.4±5.2% after 14 weeks. The fragmentation was showed at a delayed stage of the dissolution test. It may be due to the hydration of the core the PLGA-based devices which occurred after the main erosion of the matrix. Therefore, the decrease of the pH, combined with the complexity to extract mAb1 from the core, appeared more deleterious than during the main erosion process. The monomer content was at 96.5±0.3% after 24 h (burst effect), and then decreased to 74.1±3.6% and 64.6±3.3% after 6 and 12 weeks, respectively.

ELISA assays were performed to evaluate the binding capacity of mAb1 after its diffusion from the devices to the dissolution medium (see FIG. 6). The binding capacity of mAb1 was found to be 69.0±1.5% after 24 h. A slight decrease of the binding capacity was demonstrated after 5 weeks (66.2±3.8%). After 10 and 15 weeks, the binding capacity drastically decreased to 43.8±6.8% and 38.8±7.9%, respectively. Although the value after 24 hours was lower than expected in view of the low HMWS level and the high monomer content (96.5±0.3%) observed (FIG. 5), the results are very promising as they show that despite thermal stress, mAb1 is still able to bind its target, and therefore likely still active, and that continuous release can be obtained for several weeks.

### Example 7 -Stability Study of mAb1-Loaded 3DP Devices

Stability over time being an important aspect to consider when a drug product is developed, the effect of storage temperatures using 5±3° C. and 25±2° C. for 6 months (T0, T1, T2, T3 and T6 months) was assessed. 3DP devices were produced using mAb1 stabilized with the TRE-LEU combination.

Physical state of polymeric matrix: DSC analyses of the 3DP devices were compared at different time points (see Table 5). As previously mentioned, the PLGA was plasticized using PEG at 11% (w/w) and the Tg of the filament (before printing) was 21.8±0.4° C. The Tg of the reference samples (T0) were close to this value with 20.7±0.3° C. No increase of the Tg was observed over 3 months according to both storage temperatures (i.e. 5° C. and 25° C.) However, an increase of the Tg to 29.7±0.3° C. (T6) was observed after 6 months at 25° C. The Tg of devices during the stability study remained steady at 5° C. Besides, minor melting peaks were observed on samples stored at 25° C. for 2 months (T2), 3 months (T3) and 6 months (T6). The Tm were observed at 45.2±1.4° C. (T2); 45.9±0.8° C. (T3) and 46.7±0.4° C. (T6). The melting peak could be attributed to the PEG which was able to move at temperature higher than Tg of the polymeric matrix (i.e. 25° C.). The melting enthalpy of these melting peaks were recorded and showed an increase over months from 1.7±0.9 J/g (T2) to 7.4±0.6 J/g (T6). The increase of the melting enthalpy demonstrated a probably phase separation with the PLGA chain mobility at 25° C. After 2 and 3 months, the melting enthalpy remained low and the plasticizing effect was effective. The increase of the Tg after 6 months at 25° C. was associated with a higher value of melting enthalpy which was consistent with a phase separation between PLGA and PEG. The melting enthalpy of neat PEG was recorded around 193.4 J/g (Data not shown). Therefore, only a small quantity of the PEG tended to separate from the PLGA blend over 6 months. Similar observations were observed during polymer ageing study. It was reported that PEG was able to crystallize over time due to an elevation of storage temperature and humidity. The crystallization of the PEG may increase the stiffness of the devices and modify their mechanical and release properties.

TABLE 5
3DP devices printed to perform the stability study identified using the
time points (T0, T1, T2, T3, T6) with their characteristics such as
Tg (° C.), Tm (° C.), melting enthalpy (J/g), Mw (kDa).

| Storage | | | | | |
|---|---|---|---|---|---|
| temperature | Time point | | | Melting | |
| (° C.) | (months) | Tg (° C.) | Tm (° C.) | enthalpy (J/g) | Mw (kDa) |
| | T0 | 20.7 ± 0.3 | / | / | 17.2 ± 0.4 |
| 5 | T1 | 20.6 ± 1.0 | / | / | 17.1 ± 0.2 |
| | T2 | 20.2 ± 1.0 | / | / | 17.0 ± 0.1 |
| | T3 | 20.5 ± 0.2 | / | / | 17.1 ± 0.2 |
| | T6 | 20.4 ± 1.0 | / | / | 14.7 ± 0.1 |
| 25 | T1 | 20.6 ± 1.2 | / | / | 17.0± 0.1 |
| | T2 | 20.1 ± 0.9 | 45.2 ± 1.4 | 1.7 ± 0.9 | 16.7 ± 0.4 |
| | T3 | 21.3 ± 1.8 | 45.9 ± 0.8 | 2.6 ± 2.1 | 16.6 ± 0.1 |
| | T6 | 29.7 ± 0.3 | 46.7 ± 0.4 | 7.4 ± 0.6 | 13.3 ± 0.6 |

The degradation of the PLGA was assessed using GPC measurement. The Mw of T0 was recorded at 17.02±0.38 kDa which was consistent with the raw PLGA as received (Mw: 17.05±0.45 kDa). No degradation occurred over 3 months of storage. These results demonstrated the stability of the devices when they were stored at 5° C. and 25° C. for 3 months. However, the result obtained after 6 months were stored in the fridge for 1.5 months and may affect the Mw of the polymer due to the relative humidity.

Visual assessment of the devices over storage time: Visual assessment was carried out on the 3DP devices stored at 5° C. and 25° C. (not shown). No difference was observed on the devices stored at 5° C. over 6 months. Sticky specimens were observed when devices were kept at 25° C. Devices adhered to the bottom of glass vial but no loss of material was observed during the withdrawal step. This observation was performed on every device stored at 25° C. from T1 to T6. The cross-section of devices T6 at 25° C. showed high porous network due to the mobility of the chain. An increase of the device porosity was expected to have a faster release of mAb1 during the dissolution study.

Drug content and extraction from the devices: The targeted loading was 15% (w/w). As shown in Table 6, mAb1 loading in each device was consistent with the values obtained experimentally.

TABLE 6
Comparison of mAb1 loading (%), monomer content (%) and
both HMWS and LMWS levels (%) according
to the time point from T0 (reference) to T6 (6 months).

| Storage | | | | | |
|---|---|---|---|---|---|
| temperature | Time point | Loading | Monomer | HMWS | LMWS |
| (° C.) | (months) | (%) | (%) | (%) | (%) |
| | T0 | 15.4 ± 0.5 | 95.3 ± 0.2 | 4.7 ± 0.2 | 0.02 ± 0.02 |
| 5 | T1 | 15.3 ± 0.3 | 95.7 ± 0.2 | 4.3 ± 0.2 | 0.05 ± 0.05 |
| | T2 | 16.1± 0.3 | 95.9 ± 0.3 | 4.0 ± 0.3 | 0.03 ± 0.06 |
| | T3 | 16.1 ± 0.2 | 95.4 ± 0.2 | 4.6 ± 0.2 | 0 |
| | T0 | 15.4 ± 0.2 | 95.7 ± 0.1 | 4.3 ± 0.1 | 0.08 ± 0.01 |
| 25 | T1 | 15.3 ± 0.4 | 95.6 ± 0.3 | 4.4 ± 0.3 | 0 |
| | T2 | 15.5 ± 0.2 | 95.3 ± 0.3 | 4.7 ± 0.3 | 0.03 ± 0.05 |
| | T3 | 15.8 ± 0.3 | 95.7 ± 1.3 | 4.3 ± 1.3 | 0 |
| | T6 | 15.4 ± 0.3 | 94.8 ± 0.2 | 5.1± 0.2 | 0.08 ± 0.01 |

The stability of mAb1 was also assessed at each time point (Table 6). The monomer content remained stable over 6 months at 5° C. However, a slight decrease of the monomer percentage was observed after 6 months at 25° C. This decrease was associated to an increase of both HMWS (5.1±0.2%) and LMWS (0.08±0.01%) levels of the samples.

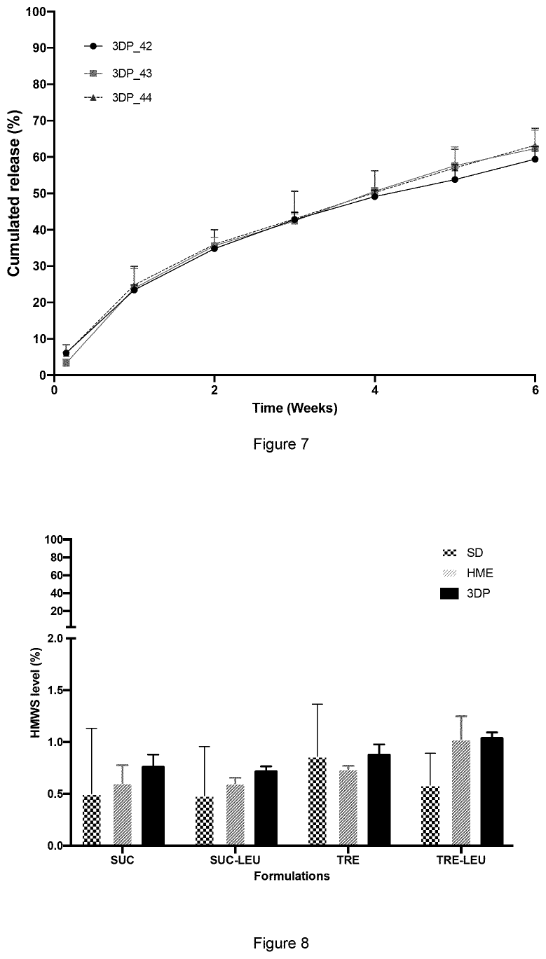

A dissolution study was performed on printed devices (3DP_39 to 3DP_44; Table 7) to investigate the release patterns as a function of the infill of the devices (FIG. 7). The burst release from all devices were limited regardless the formulation. For instance, the burst release of 3DP_42 reached 6.1±0.5% after 24 h. As previously observed, triphasic profiles were observed with all devices. These results gave consistency to the previous results showed on mAb1-loaded 3DP devices (FIG. 4a). Finally, a maximum cumulated release of 63.2±4.7% and 62.3±5.1% with the devices 3DP_43 and 3DP-44 was demonstrated after 6 weeks. As it can be observed the in-fill of the devices only slightly impact the cumulative release of the antibody.

TABLE 7
HME batches starting material and associated 3DP batches printed
with an infill density of 10, 50 or 100% (v/v) and 0.3 mm of
layer thickness (n = 3).

| HME batch number | 3DP batch number | | |
|---|---|---|---|
| (starting material; see | Infill (% (v/v) | | |
| Table 1) | 10 | 50 | 100 |
| HME_18 | 3DP_39 | 3DP_40 | 3DP_41 |
| HME_19 | 3DP_42 | 3DP_43 | 3DP_44 |

### Example 8—Filaments and 3DP Devices Containing fAb2

Based on the findings from examples 1 to 7, TRE and SUC as stabilizers, with and without the addition of LEU, were investigated on fAb2, a Fab antibody fragment.

A similar approach was applied to fAb2 with successive processing methods such as buffer exchange (BE), spray-drying (SD), Hot melt extrusion (HME) and 3D printing (3DP) at the end. The formulations of fAb2 were made of TRE or SUC with or without LEU to compare four different formulations and find out which one was able to stabilize the Fab against thermal stresses (Table 8) and associated 3D printing (3DP) batches with layer thickness of 0.1 mm and 0.3 mm. fAb2 was at 8% w/v in the initial liquid composition, 66.7% w/w in the SD powder and 15.3% w/w in the filaments/3DP devices.

Characteristics of the extracted fAb2: The raw fAb2 material was characterized with a high monomer content of 99.6±0.2% and a low HMWS level of 0.4±0.2%. The extracted fAb2 from the PLGA matrix after HME and 3DP were compared with the extracted fAb2 from SD powder (FIG. 8). The HMWS levels of the formulated Fab were slightly increased during the high temperature processes (e.g. HME and 3DP). For instance, the HMWS level of formulation containing TRE-LEU evolved from 0.6±0.3% (SD) to 1.0±0.1% after 3DP. According to the all results, none of them were significantly different from the Fab-SD powder (p-value>0.05).

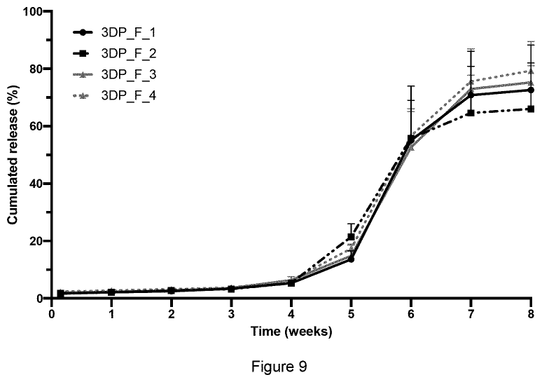

Dissolution study: A dissolution study was performed on all printed devices (DDS; 3DP-F1 to 3DP_F4) to investigate both the release patterns and the stability of the Fab over time (FIG. 9). The burst release from all devices were limited regardless the formulation. For instance, the burst release of 3DP_F4 reached 2.4±0.2% after 24 h. As previously observed, triphasic profiles were observed with all devices. This observation was mainly dependent on the monolithic state of the device and the difficulty of water penetration. Consequently, a faster release was observed between the week 5 and week 6. These results gave consistency to the previous results showed on mAb1-loaded 3DP devices (FIGS. 4a and 7). Finally, a maximum cumulated release of 79.3±1.7% with the devices 3DP_F4 was demonstrated after 8 weeks.

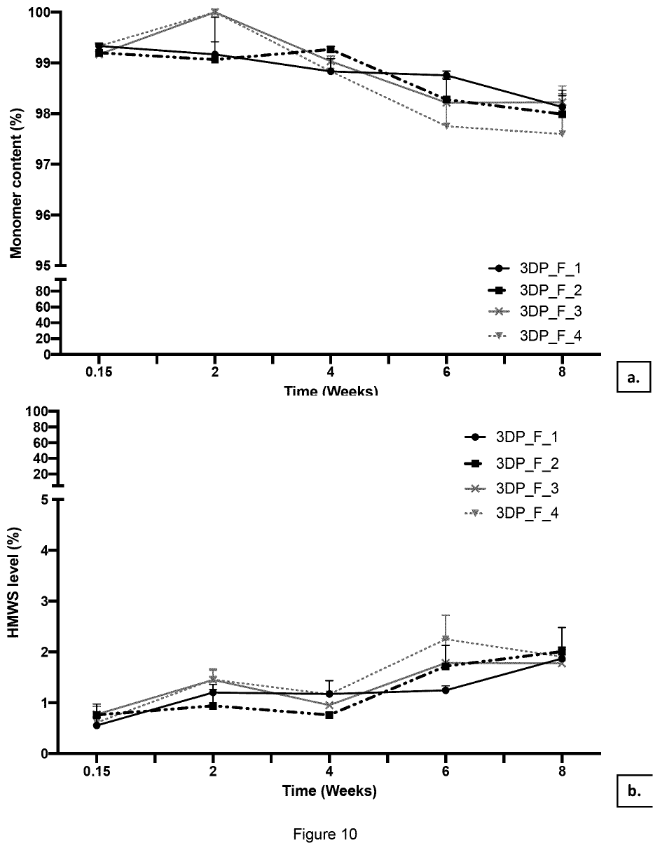

Stability study of fAb2 over time: During the dissolution test, both monomer content and HMWS level were investigated over 8 weeks (FIG. 10). A slight decrease of monomer content was observed after 8 weeks. Indeed, the monomer content evolved from 99.3±0.1% to 97.6±1.0% with 3DP_F4 (FIG. 10a). After 2 weeks of dissolution medium, a deformation of the monomer peak on the thermogram was observed (data not shown). A shoulder appeared on the monomer peak and increased over weeks, but no fragmentation was reported after 8 weeks, regardless the formulation (data not shown). This occurrence on the chromatogram may be related to the acidic microclimate pH in the PLGA matrix which impaired the Fab integrity. An increase of the HMWS level was demonstrated over the dissolution time (FIG. 10b). The aggregation of fAb2 over dissolution weeks seemed limited in comparison with the previous results generated on mAb1. For instance, the value of 3DP_F4 after 8 weeks of dissolution was 1.9±0.1%. No fragmentation of fAb2 was showed according to the dissolution time of 8 weeks. FAb2 seemed to be more stable when similar conditions were applied on. Indeed, the aggregation of fAb2 over weeks was quite low after high temperatures processes and during the dissolution test.

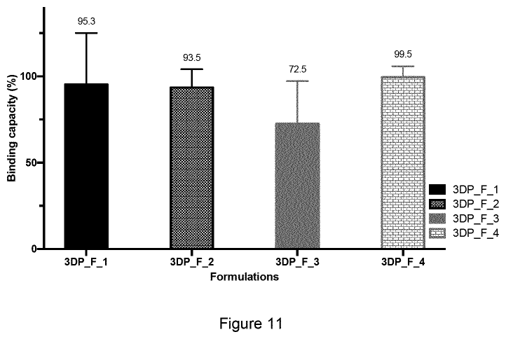

Binding capacity study: The binding capacity of fAb2 was assessed to confirm that it was still able to bind its target. ELISA test demonstrated that the binding capacity of fAb2 was preserved after 24 h of release, regardless the formulation. For instance, the binding capacity of 3DP_F4 was 99.5±6.4% (FIG. 11). These results suggested that all formulations were adapted to successively dry the Fab, produce a printable filament and print the 3DP devices with a limited degradation of the Fab.

Conclusion: fAb2 was successively dried, extruded and 3D printed using four different formulations containing SUC or TRE with/without LEU. The 3DP DDS allowed a sustained release of the Fab over at least 8 weeks. The HMWS level remained quite low with a maximum value of 1.9±0.1% (3DP_F4). Although results obtained with formulations comprising TRE (+/−LEU) as stabilizers were slightly better in term of total release, SUC (+/−LEU) were also very promising.

### Overall Conclusion

The results herein presented surprisingly showed for the first time that not only HME but also the association of HME and FDM 3D printing were suitable to produce antibody-loaded filaments and antibody-loaded implantable devices in which the antibody can still bind its target (and therefore is likely still active), as herein shown with a monoclonal antibody (mAb1) and a Fab fragment (fAb2). Homogeneous solid dispersion of the antibodies in the PLGA matrix was reached in both printable filaments and 3DP devices. Different stabilizers were investigated to stabilize the antibodies against thermal degradation. The most promising ones (trehalose and sucrose) promoted the mAb integrity during SD, HME and 3DP steps using different mAb: stabilizer ratios. The further optimization of the formulation, using a low amount of an amino acid, such as Leucine, led to improved stability of the antibodies against potential thermal degradation. In addition, dissolution profile demonstrated an interesting sustained-release profile with a limited burst effect, especially with an antibody fragment. Finally, it was demonstrated that, despite the relatively high temperatures of extrusion (90° C.) and printing (105° C.), the binding capacity of the antibodies remains for about at least 5 weeks.

TABLE 8
Theoretical composition of evaluated fAb2 formulations for SD batches (% w/V), solid
composition of SD powders (% w/w) and the yield of the spray-drying process (%), printable
filaments produced using HME batches (% w/w) with the yield of the process (%) and associated
3DP batches printed with an infill density of 100% (v/v) and 0.3 mm of layer thickness (n = 3).

| | | | | | | | | | SD | | RG502 | PEG | Excip. | FAD | | |
|---|---|---|---|---|---|---|---|---|---|---|---|---|---|---|---|---|
| | | | HIS | Stab | LEU | | | | yield | | (% | (% | (% | (% | | 3DP |
| | | | liquid | | | HIS | Stab. | LEU | (%) | | w/w) | w/w) | w/w) | w/w) | | 0.3 |
| SD | | fAb: | composition | | | Solid composition | | | | HME | Filament composition | | | | HME | mm |
| batch | | stab | (% w/v) after | | | (% w/w) | | | | batch | (% w/w) after | | | | yield | batch |
| No. | Stab. | ratio | buffer exchange | | | after SD | | | | No. | HME | | | | (%) | No. |
| SD F1 | SUC | 2.0:1 | 0.2 | 3.8 | — | 1.9 | 31.4 | — | 87.9 | HME_F1 | 69.4 | 7.6 | 7.6 | 15.3 | 58.3 | 3DP_F1 |
| SD_F2 | SUC- | | 0.2 | 3.2 | 0.6 | 1.9 | 26.4 | 5.0 | 91.1 | HME_F2 | 69.4 | 7.6 | 7.6 | 15.3 | 57.8 | 3DP_F2 |
| | LEU | | | | | | | | | | | | | | | |
| SD_F3 | TRE | | 0.2 | 3.8 | — | 1.9 | 31.4 | — | 85.5 | HME_F3 | 69.4 | 7.6 | 7.6 | 15.3 | 56.0 | 3DP_F3 |
| SD_F4 | TRE- | | 0.2 | 3.2 | 0.6 | 1.9 | 26.4 | 5.0 | 89.5 | HME_F4 | 69.4 | 7.6 | 7.6 | 15.3 | 55.9 | 3DP_F4 |
| | LEU | | | | | | | | | | | | | | | |

Excip. = excipients

### REFERENCES

1. Goyanes et al. (2015), Mol. Pharmaceutics 2015, 12:4077-4084
2. Tiwari et al. (2016) Expert Opinion On Drug Delivery, 13 (3):451-464
3. Fredenberg et al. (2011), International Journal of Pharmaceutics, 415:34-52
4. Cossé et al. (2016), AAPS PharmSciTech., 18:15-26
5. Duque et al. (2018), International Journal of Pharmaceutics, 538:139-146
6. Ghalanbor et al. (2010), Pharmaceutical Research, 27 (2):371-379
7. Jamroz et al. (2018), Pharm. Res., 35: 176
8. Azad et al. (2020), Pharmaceutics, 12 (2), 124
9. Norman et al. (2017), Advanced Drug Delivery Reviews, 108:39-50
10. Jamróz et al. (2018), Pharm. Res., 35:176
11. Mensink et al. (2017), European Journal of Pharmaceutics and Biopharmaceutics, 114:288-295
12. Diwan and Park (2001), J Control Release, 73 (2-3):233-44
13. White et al. (2013), Mater Sci Eng C Biol Appl., 33 (5):2578-83.
14. Crowley et al. (2007), Drug Development and Industrial Pharmacy, 33:909-926
15. WO2015/197772
16. Sadia et al. (2016), International Journal of Pharmaceutics, 513 (1-2): 659-668
17. Arrighi et al. (2019), International Journal of Pharmaceutics, 566:291-298
18. Marquette et al. (2014), European Journal of Pharmaceutics and Biopharmaceutics, 86:393-403
19. Pignatello et al. (2009), Nanomedicine, 4 (2):161-175
20. Zhang et al. (2017), International Journal of Pharmaceutics, 519:186-197
21. Le Basle et al. (2020), Journal of Pharmaceutical Sciences, 109:169-190
22. Wang et al. (2007) Journal Of Pharmaceutical Sciences, 96 (1): 1-26
23. Baek et al. (2017), Pharm. Res., 34:629-639
24. Bowen et al. (2013), Drying Technology, 31:1441-1450
25. Gidwani and Vyas (2015), BioMed Research International, vol. 2015, Article ID 198268
26. Kanojia et al. (October 5, 2016) PLOS ONE, DOI:10.1371/journal.pone.0163109
27. Maury et al. (2005), European Journal of Pharmaceutics and Biopharmaceutics, 59:251-261
28. Melocchi et al. (2015), Journal of Drug Delivery Science and Technology, 30:360-367
29. Carlier et al. (2019), International Journal of Pharmaceutics, 565:367-377
30. Minne et al. (2008), European Journal of Pharmaceutics and Biopharmaceutics, 70:839-844
31. Ghalanbor et al. (2013), European Journal of Pharmaceutics and Biopharmaceutics, 85:624-630

1-17. (canceled)

18. A filament for preparing an implantable drug delivery device, wherein the filament comprises at least one polymeric material, a plasticizer and an active ingredient, wherein said active ingredient is an antibody.

19. The filament according to claim 18, wherein the filament further comprises at least one stabilizer, a buffering agent and/or a surfactant.

20. The filament according to claim 18, wherein the at least one polymeric material is poly(lactic-co-glycolic acid) (PLGA), poly(ε- caprolactone) (PCL), poly(lactic acid) (PLA), or combinations thereof.

21. The filament according to claim 18, wherein the at least one polymeric material is in a range of about 50 to 75% (w/w).

22. The filament according to claim 18, wherein the plasticizer is polyethylene glycol.

23. The filament according to claim 18, wherein the plasticizer is in a range of about 2 to 20% (w/w).

24. The filament according to claim 19, wherein the at least one stabilizer is a disaccharide, cyclic oligosaccharides, polysaccharides, a polyol, or an amino acid, or any combinations thereof and wherein said stabilizer is in an amount within the range of about 5 to 15% (w/w).

25. The filament according to claim 24, wherein the disaccharide is sucrose or trehalose, the cyclic oligosaccharide is hydroxypropyl-β-cyclodextrin, the polysaccharide is inulin, the polyol is sorbitol, the amino acid is L-Arginine, L-Leucine, L-phenylalanine or L-Proline.

26. The filament according to claim 18, wherein the active ingredient is homogeneously dispersed into the polymeric matrix.

27. The filament according to claim 18, wherein the active ingredient loading is in the range of 15 to 35% (w/w).

28. The filament according to claim 19, wherein the ratio antibody: stabilizer is between 1:1 and 5:1 (w/w).

29. An implantable drug delivery device comprising one or more layer(s) made from a filament according to claim 18.

30. The implantable drug delivery device according to claim 29, wherein the device is printed using a layer thickness of 100 μm to 400 μm.

31. The implantable drug delivery device according to claim 29, wherein the device comprises at least one internal hollow cavity.

32. The implantable drug delivery device according to claim 29, wherein the device is a fully solid object.

33. A 3D printed implantable drug delivery device obtained by 3D printing a filament according to claim 18.

34. A process for producing a filament according to claim 18, the process comprising the steps of:
a) preparing a liquid formulation comprising the active ingredient, wherein said liquid formulation may further comprise at least one stabilizer, a buffering agent and/or a surfactant,
b) freeze-drying or spray-drying the liquid formulation of step a) to obtain dry microparticles,
c) dispersing homogeneously the dry microparticles of step b) with a plasticizer and at least one polymeric material,
d) extruding the dispersion of step c) by hot melting extrusion (HME) to obtain a filament.

35. A process for producing the implantable drug delivery device, the process comprising the steps of:
a) loading a filament according to claim 18 into the print head of the 3D printer using a temperature above the glass transition temperature of the polymeric material of matrix,
b) heating the build platform at a temperature below the glass transition temperature of the polymeric matrix,
c) depositing said heated a filament through a nozzle to build the device from at least the first layer to the final top layer.
